(19) United States
(12) Patent Application Publication
(10) Pub. No.: US 2021/0401678 A1
(43) Pub. Date: Dec. 30, 2021

(54) COSMETIC COMPOSITION CONTAINING INORGANIC PARTICLES FOR BLOCKING BLUE LIGHT

(71) Applicant: COSMAX, INC., Gyeonggi-do (KR)

(72) Inventors: Su Ji KIM, Gyeonggi-do (KR); Ji Hyun LEE, Gyeonggi-do (KR); Jun Bae LEE, Gyeonggi-do (KR); Myeong Sam PARK, Gyeonggi-do (KR)

(21) Appl. No.: 17/288,970

(22) PCT Filed: Sep. 5, 2019
(86) PCT No.: PCT/KR2019/011503

§ 371 Date: Apr. 27, 2021

(30) Foreign Application Priority Data
Oct. 29, 2018 (KR) 10-2018-0129959

(51) Int. Cl. A61K 8/02; A61K 8/25; A61Q 17/04; A61Q 19/08

(52) U.S. Cl. CPC A61K 8/0279; A61K 8/25; A61K 2800/412; A61Q 19/08; A61K 2800/26; A61Q 17/04

(57) ABSTRACT

The present invention relates to a cosmetic composition comprising inorganic particles for blocking blue light, containing inorganic particles having a blue light blocking effect as an active ingredient in the composition, reflecting and scattering light in a blue light wavelength range, thereby significantly blocking light of a blue light area that has otherwise been difficult to block conventionally.

### TECHNICAL FIELD

The present invention relates to a cosmetic composition comprising inorganic particles for blocking blue light.

### BACKGROUND ART

The light we see in our daily lives is visible light, which is an electromagnetic wave with a wavelength in a range of approximately 380 to 800 nm. Recently, research results have been published that among these electromagnetic waves, “blue light”, which is blue light in a range of 380 to 500 nm, has an adverse effect on the skin. A paper published in 2012 (J. Invest. Dermatol., (2012) 132, 1901-1907) reported that visible light including blue light also causes premature skin aging due to the production of ROS, the expression of MMP-1, and the like similarly to ultraviolet rays, and, a paper published in 2017 (Free Radic. Biol. Med., (2017) 108, 300-310) also reported that blue light also generates active oxygen such as superoxide in keratinocytes, causing a skin aging phenomenon similar to UVA. Further, the paper in 2010 (J. Invest. Dermatol., (2010) 130, 259-269) announced that long-term exposure to blue light causes phenomena such as growth and decreased differentiation of keratinocytes.

Since blue light has the second highest energy after ultraviolet rays, long-term exposure to blue light may be a considerable burden on the eyes and body. In particular, since smart devices such as tablet PCs and smartphones are used for a very long time in human life as the main reason that blue light is regarded as a problem, it is considered that a problem with the harmful effects of blue light becomes increasingly serious. In fact, many studies on eye fatigue and dry eye syndrome caused by blue light have been conducted, and various attempts such as the production of a blue light blocking function for smartphones have been made as countermeasures against these eye fatigue and dry eye syndrome. Further, blue light weakens the secretion of melatonin which is a sleep-inducing hormone, resulting in sleep disorders, which disturbs the biological clock, so that the skin may become rough or dull.

We are exposed to light sources of blue light for a very long time due to changes in the daily living environment in which smart devices such as smartphones, tablet PCs, and computer monitors have become indispensable. Therefore, there is a need for developing cosmetics capable of blocking blue light. Korean Patent Application Laid-Open No. KR10-2017-0003391 discloses a cosmetic composition containing jade powder to block blue light, but the jade powder has problems in that the jade powder feels heavy to use as an inorganic powder and is difficult to use for the sungel type, and it is difficult to uniformly block short-wavelength blue light when the jade powder is not uniformly dispersed due to the blocking of blue light via blue light scattering reflection.

Thus, the present inventors discovered a material having a blue light blocking effect, developed a formulation containing the material, and confirmed that when the formulation was applied to the skin, the formulation had a blocking effect in the blue light area, thereby completing the present invention.

### SUMMARY

### Technical Problem

Blue light, which belongs to the visible light area, has recently emerged as a new light source that needs to be protected from the skin along with ultraviolet rays and infrared rays due to changes in the daily living environment. However, since an inorganic powder used as an ultraviolet and infrared blocking agent in the related art cannot effectively block blue light, there is also a need for a new blocking agent for blocking blue light from the skin and an evaluation method capable of evaluating the new blocking agent. Therefore, an object of the present invention is to discover inorganic particles for blocking blue light formed in a hollow spherical shape having a predetermined thickness, and to provide a cosmetic composition containing the inorganic particles for blocking blue light. Further, another object thereof is to measure the blocking effect of blue light by actually applying the cosmetic composition to the skin.

### Technical Solution

The present invention provides a cosmetic composition for blocking blue light, including inorganic particles having a hollow structure as an active ingredient.

The present invention provides a cosmetic composition for blocking blue light, in which the inorganic particles having a hollow structure are silica. Here, the inorganic particles have a particle size of 1 to 100 μm, preferably 1 to 20 μm. The present invention provides a cosmetic composition for blocking blue light, in which the inorganic particles are present in an amount of 0.001 to 20 wt %, preferably 0.01 to 10 wt %, based on a total weight of the composition.

The present invention also provides a cosmetic composition for blocking blue light, which is formulated into any one form of moisture gel, moisture cream, emulsion, serum, lotion, cream, sun cream, stick sun cream, liquid foundation and solid foundation.

Meanwhile, the present invention provides a method for preparing a cosmetic composition for blocking blue light, the method including: preparing a water phase part; preparing an oil phase part including inorganic particles for blocking blue light; and mixing the oil phase part with the water phase part.

### Advantageous Effects

The cosmetic composition for blocking blue light according to the present invention contains inorganic particles having a blue light blocking effect as an active ingredient to significantly block light in the blue light area, which has been difficult to block in the related art by reflecting and scattering light in the blue light wavelength range, thereby having an effect of preventing photo aging.

### BRIEF DESCRIPTION OF THE DRAWINGS

FIG. 1 illustrates a measurement graph of the spectral reflectance of inorganic particles used as ultraviolet materials.

FIGS. 2A and 2B are photographs for illustrating a scanning electron microscope measurement photograph for silica having a hollow structure and closed structure.

FIG. 3 illustrates a graph comparing the spectral reflectances of a cosmetic composition containing inorganic particles for blocking blue light and a cosmetic composition containing no inorganic particles for blocking blue light in the blue light area.

### DETAILED DESCRIPTION

Hereinafter, the present invention will be described in detail such that a person skilled in the art to which the present invention pertains can easily carry out the present invention with reference to the accompanying techniques. However, it will be obvious to those skilled in the art that the present invention can be implemented in various different forms, and is not limited to the Examples described herein.

The present invention is a cosmetic composition for the purpose of blocking blue light, and the cosmetic composition for blocking blue light of the present invention blocks blue light from reaching the skin by reflecting and scattering light in the blue light wavelength range.

Unlike ultraviolet rays, blue light does not cause visible skin changes such as erythema and darkening when the skin is irradiated with blue light. Therefore, it is impossible to apply an existing UV blocking clinical evaluation method to a blue light blocking evaluation method. Light that enters the skin spectroscopically has three paths of reflection, absorption, and transmission, and among them, the spectroscopic physical quantity of light that can be measured from the actual skin is a quantity for reflection. That is, this is because it is impossible to measure the degree of light absorption or transmission when a cosmetic formulation is applied to human skin, but it is possible to measure the reflectance of the skin before and after the application. Accordingly, the present invention developed a clinical evaluation method focusing on how much blue light is reflected by the skin in the blue light wavelength area. Meanwhile, hollow spherical particles containing an organic ultraviolet blocking agent have already been published, but the organic blocking agent has a property of absorbing light, and thus is not suitable for objectively determining whether the organic blocking agent has a blue light blocking effect.

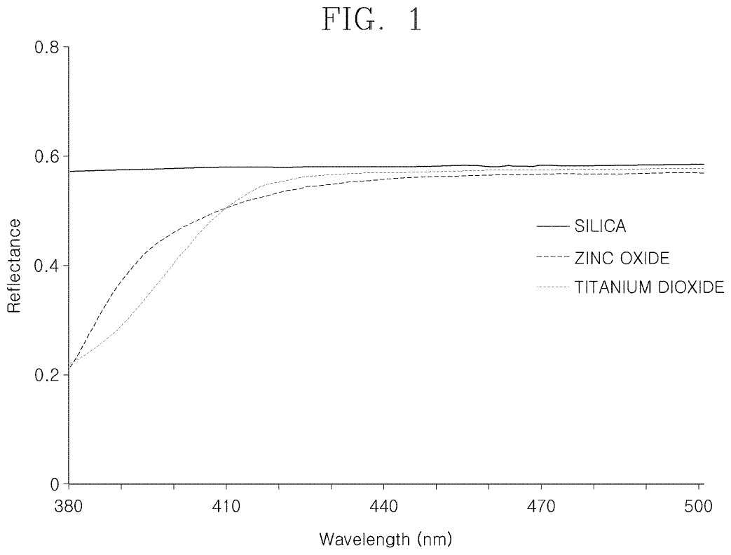

Before blue light blocking inorganic particles are applied to a composition, among inorganic materials used as active ingredients of an ultraviolet blocking agent in the related art, the spectral reflectances of titanium dioxide, zinc oxide, and silica themselves in the blue light region were measured by a spectrophotometer (ASD Inc., USA) and are shown in FIG. 1. The higher the spectral reflectance, the higher the ability to block blue light.

As a result, it could be seen that the spectral reflectance of silica was the highest compared to that of titanium dioxide or zinc oxide. In particular, referring to FIG. 1, silica exhibited an excellent reflectance in the vicinity of the high energy region even in blue light. Thus, the present inventors used a silica material as the inorganic particles of the composition of the present invention.

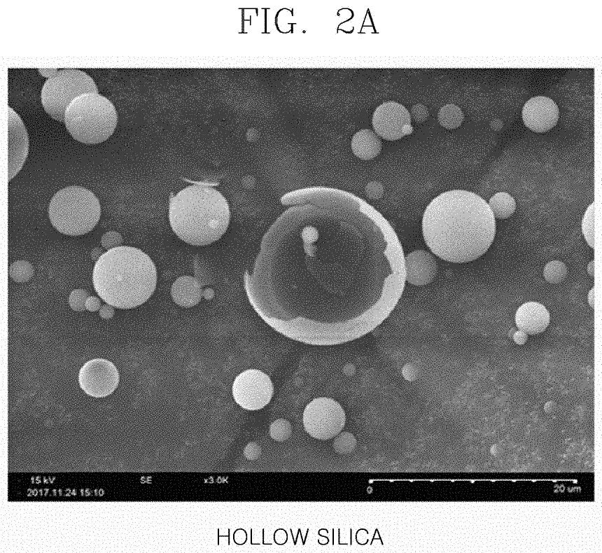

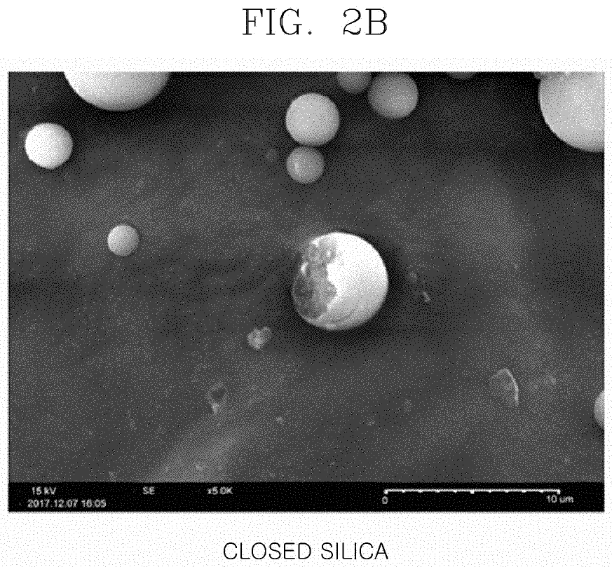

The composition of the present invention contains silica having a hollow structure therein as an active ingredient, and it can be confirmed that the internal space of the spherical particles is hollow when observed with a scanning electron microscope (see FIG. 2A). Referring to FIGS. 2A and 2B, it can be confirmed that hollow silica has a size of 1 to 20 μm and the inside of the particles thereof is empty (FIG. 2A), and it can be confirmed that closed silica has a size of 1 to 10 μm and the inside thereof is packed (FIG. 2B). The hollow structure has an empty inside unlike the closed structure, so that the hollow structure is more advantageous in blocking blue light than the closed structure because the reflection in the external representation of the particle and the scattering effect in the inside simultaneously occur.

The hollow silica inorganic particles of the present invention have a particle size of 1 to 100 μm, more preferably 1 to 20 μm, and preferably not more than 100 μm in consideration of dispersibility, transparency, usability, and the like.

In the present invention, the amount of the hollow silica inorganic particles can be easily determined by the person skilled in the art within a range that does not impair the purpose and effect of the present invention, but includes preferably about 0.001 to 20 wt %, more preferably about 0.01 to 10 wt %, based on the total weight of the composition of the present invention. When the amount exceeds 20 wt % based on the total weight of the composition, the usability may deteriorate.

The cosmetic composition for blocking blue light according to the present invention can be prepared in any formulation typically prepared in the art. For example, the product form may be a moisture gel, moisture cream, emulsion, serum, lotion, cream, sun scream, stick sun cream, liquid foundation, solid foundation formulation or the like.

In addition, the cosmetic composition for blocking blue light of the present invention may also be used as a product having multiple functions by being used in combination with a material for blocking ultraviolet rays and infrared rays.

Hereinafter, the present invention will be described in more detail through Examples. However, these Examples are provided only for exemplarily explaining the present invention, and the scope of the present invention is not limited by these Examples.

### EXAMPLES

1. Preparation of Cosmetic Composition

### Example 1. Preparation of Cosmetic Composition for Blocking Blue Light, Containing 2.5 wt % of Inorganic Particles for Blocking Blue Light

The cosmetic composition of Example 1 was prepared as the composition shown in the following Table 1. 2.5 wt % of inorganic particles for blocking blue light were mixed and dispersed in the oil phase part shown in Table 1, and then warmed and dissolved at 70 to 80° C. Thereafter, the cosmetic composition was prepared by adding a water phase part thereto and stirring the resulting mixture. The inorganic particles of Example 1 of the present invention are hollow spherical silica particles.

In order to verify the blue light blocking effect of the composition of Example 1, the spectral reflectance spectrum in a region of 380 to 500 nm was measured using a spectrophotometer (ASD Inc., USA) capable of measuring a spectral reflectance at a wavelength of 350 to 2500 nm.

TABLE 1

| | | Content (wt %) | | |
|---|---|---|---|---|
| | | Compara- | | |
| | | tive Exam- | Exam- | Exam- |
| | Ingredient | ple 1 | ple 1 | ple 2 |
| Oil | Lauryl PEG-polydimethyl- | 1.00 | 1.00 | 1.00 |
| phase | siloxyethyl dimethicone | | | |
| | PEG-10 Dimethicone | 3.00 | 3.00 | 3.00 |
| | Butylene glycol | 3.00 | 3.00 | 3.00 |
| | dicaprylate/dicaprate | | | |
| | Diphenylsiloxy phenyl | 2.00 | 2.00 | 2.00 |
| | trimethicone | | | |
| | Cyclopentasiloxane | 5.00 | 5.00 | 5.00 |
| | Cyclohexasiloxane | 5.00 | 5.00 | 5.00 |
| | Dimethicone | 8.00 | 8.00 | 8.00 |
| | Disteardimonium | 0.50 | 0.50 | 0.50 |
| | hectorite | | | |
| Inorganic | Silica (closed) | 2.50 | — | — |
| Material | Silica for blocking | — | 2.50 | 5.00 |
| | blue light (hollow) | | | |
| Water | Purified water | To 100 | To 100 | To 100 |
| phase | | | | |
| | Disodium EDTA | 0.02 | 0.02 | 0.02 |
| | Polyol | 5.00 | 5.00 | 5.00 |
| | Preservative | Appro- | Appro- | Appro- |
| | | priate | priate | priate |
| | | amount | amount | amount |
| Total | | 100 | 100 | 100 |

### Example 2. Preparation of Cosmetic Composition for Blocking Blue Light, Containing 5.0 wt % of Inorganic Particles for Blocking Blue Light

The cosmetic composition of Example 2 was prepared as the composition shown in Table 1. Specifically, 5.0 wt % of inorganic particles for blocking blue light were mixed and dispersed in the oil phase part in the same manner as in the method described in Example 1, and then warmed and dissolved at 70 to 80° C. Thereafter, the cosmetic composition was prepared by adding a water phase part thereto and stirring the resulting mixture. The inorganic particles of Example 2 of the present invention are hollow spherical silica particles, and the difference from Example 1 is that the inorganic particles are mixed in an amount of 5.0 wt %.

Examples 1 and 2 were prepared to compare the blue light blocking effects according to the contents of the existing ultraviolet blocking inorganic material and the inorganic particles for blocking blue light.

### Comparative Example 1. Preparation of Cosmetic Compositions Containing Existing Ultraviolet Blocking Inorganic Particles

The cosmetic composition of Comparative Example 1 was prepared as the composition shown in Table 1. Specifically, an inorganic material was mixed with an oil phase part and dispersed in the oil phase part, and then the resulting dispersion was warmed to 70 to 80° C. Thereafter, the cosmetic composition was prepared by adding a water phase part thereto and stirring the resulting mixture.

The blue light blocking effect was compared with that of the composition of the present invention containing hollow silica, using closed silica as the inorganic particles of Comparative Example 1.

2. Performance Test

### Experimental Example 1. Evaluation of Human Body In Vivo Blocking Effect of Cosmetic Composition for Blocking Blue Light

In order to evaluate the blue light blocking effect of the emulsions prepared in the Comparative Example and the Examples, the following evaluation method developed in-house was used. The blue light blocking effect was evaluated by comparing the spectral reflectances before and after the emulsion application at a wavelength in a blue light area, and the apparatus used for the measurement was the same as that used in Experimental Example 1.

[Method of Evaluating Blue Light Blocking Effect]

1) Preparation with a size of 3.5×4 cm on the inner skin of the human arm

2) Measurement of spectral reflectance before application of sample (control)

3) Uniform application of sample of 2 μL/cm² using fingers

4) Allowing the sample to stand for 15 minutes

5) Measurement of spectral reflectance of the place to which sample is applied (sample)

6) Measurement of blue light blocking effect by substituting the spectral reflectances before and after application of sample into the following Equation 1 (BPF: Blue-light Protection Factor)

$$BPF = \frac{\begin{matrix}{\int }_{380 nm}^{500 nm} Reflectance ( after ) - \\ {\int }_{380 nm}^{500 nm} Reflectance ( before )\end{matrix}}{{\int }_{380 nm}^{500 nm} Reflectance ( before )} \times 100 \qquad [ Equation 1 ]$$

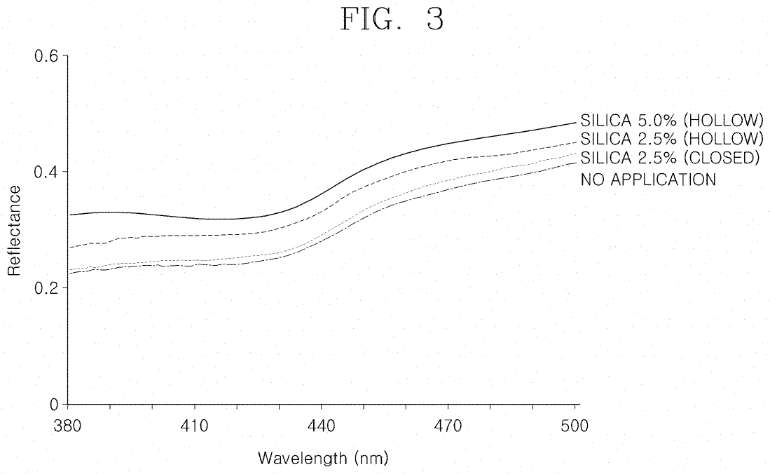

The blue light blocking effects of Comparative Example 1 and Examples 1 and 2 are compared, and the results thereof are shown in FIG. 3. It can be determined that the more wavelengths in the blue light area are reflected, the better the blue light blocking effect is, and accordingly, it was compared how much the spectral reflectance after application was higher than that before the sample was applied.

In FIG. 3, no application is a measurement result of the spectral reflectance of the skin to which the product is not applied, and the rest is a measurement result of three cosmetic compositions prepared according to the composition of Table 1. It can be seen that Comparative Example 1 is a blocking agent containing closed silica used as an existing material for blocking ultraviolet rays, and fails to effectively block blue light because the difference in spectral reflectance before and after applying the cosmetic is very small.

It can be seen that the case where compositions (Example 1, Example 2) prepared by including a hollow silica, which is a blue light blocking inorganic material, is applied has a much higher spectral reflectance than before the cosmetic is applied in the blue light area and the case where a composition including closed silica is applied. This indicates that hollow silica is more effective for blocking blue light from the skin by reflecting and scattering blue light more than closed silica, which is an existing inorganic material that blocks ultraviolet rays.

### Experimental Example 2. Evaluation Test of Usability of Cosmetic Composition

The following usability was evaluated for 20 adult men and women who did not have skin diseases with respect to the compositions prepared in Example 1 and Comparative Example 1 containing the same content (2.5 wt %) of inorganic materials. Example 1 and Comparative Example 1 were used on the cheeks of the face, and the usability such as white cast, the degree of stiff spreadability and general satisfaction that may appear when the inorganic material was used was evaluated, and the results are shown in the following Table 2. When 15 or more people were satisfied, it was denoted as ⊚, when 10 or more people were satisfied, it was denoted as ◯, and when 5 or more people were satisfied, it was denoted as Δ.

TABLE 2

| | Comparative Example 1 | Example 1 |
|---|---|---|
| Degree of white cast | ◯ | ⊚ |
| Spreadability | ◯ | ⊚ |
| General satisfaction | ◯ | ⊚ |

Generally, in the case of a composition containing an inorganic material, titanium dioxide or zinc oxide typically has stiff usability, or when applied to the skin, the degree of white cast is severe, so that improvement is required for stiff usability and white cast. Silica has a lower degree of white cast than titanium dioxide and zinc oxide, and thus is easily used, but as shown in Table 2, it can be seen that the cosmetic composition containing hollow silica according to the present invention shows excellent usability in terms of white cast or spreadability compared to the case of using existing closed silica. As a result, it could be confirmed that the formulation containing hollow silica was excellent in satisfaction with the general usability.

1-7. (canceled)

8. A method for preparing a cosmetic composition for blocking blue light, the method comprising: preparing a water phase part;
preparing an oil phase part comprising inorganic particles for blocking blue light; and
mixing the oil phase part with the water phase part.

9. The method of claim 8, wherein the inorganic particles are comprised in an amount of about 0.001 to about 20 wt % based on a total weight of the composition.

10. The method of claim 8, wherein the inorganic particles are comprised in an amount of about 0.01 to about 10 wt % based on a total weight of the composition.

11. A method for blocking blue light, comprising:
applying a cosmetic composition comprising inorganic particles having a hollow structure to a subject.

12. The method of claim 11, wherein the inorganic particles are silica.

13. The method of claim 11, wherein the inorganic particles have a particle size of 1 to 100 μm.

14. The method of claim 11, wherein the inorganic particles have a particle size of 1 to 20 μm.

15. The method of claim 11, wherein the inorganic particles are present in an amount of 0.001 to 20 wt % based on a total weight of the composition.

16. The method of claim 11, wherein the inorganic particles are present in an amount of 0.01 to 10 wt % based on a total weight of the composition.

17. The method of claim 11, wherein the cosmetic composition is formulated into any one form of moisture gel, moisture cream, emulsion, serum, lotion, cream, sun cream, stick sun cream, liquid foundation and solid foundation.

18. The method of claim 11, wherein the subject is a human being.

19. The method of claim 11, wherein the cosmetic composition further comprises one or more ingredients selected from the group consisting of lauryl PEG-polydimethylsiloxyethyl dimethicone, PEG-10 dimethicone, butylene glycol dicaprylate, diphenylsiloxy phenyl trimethicone, cyclopentasiloxane, cyclohexasiloxane, dimethicone, and disteardimonium hectorite.
